(19) United States
(12) Patent Application Publication
(10) Pub. No.: US 2011/0318289 A1
(43) Pub. Date: Dec. 29, 2011

(54) Methods and Compositions for Reducing Body Odor

(75) Inventors: Michael Frodyma, Saskatchewan (CA); Sarah McHatton, Wake Forest, NC (US); Xianxian Liu, Sunnyvale, CA (US); Jonathan Leder, Montvale, VA (US); Kevin Mann, Roanoke, VA (US)

(73) Assignee: Novozymes Biologicals, Inc., Salem, VA (US)

(21) Appl. No.: 13/167,501

(22) Filed: Jun. 23, 2011

Related U.S. Application Data
(60) Provisional application No. 61/358,246, filed on Jun. 24, 2010.

(51) Int. Cl. A61K 8/99; A61Q 15/00; C12N 1/20

(52) U.S. Cl. 424/65; 435/252.5

(57) ABSTRACT

The invention relates to biocontrol bacteria and related methods and compositions for control of body malodor-causing bacterial species or the production of odor-causing substances by such organisms.

### CROSS-REFERENCE TO RELATED APPLICATIONS

This application claims priority or the benefit under 35 U.S.C. 119 of U.S. provisional application no. 61/358,246 filed Jun. 24, 2010, the contents of which are fully incorporated herein by reference.

### FIELD OF THE INVENTION

The present invention relates generally to biocontrol of odor-causing bacteria, e.g., in personal care products, to reduce body odor.

### BACKGROUND

The main cause of body odor is bacterial production of volatiles. In moist places on the body Corynebacterium together with Staphylococcus dominate. Body odor is mainly a by-product of bacterial degradation of sweat. Fresh sweat is odorless. There are two types of sweat glands:

Eccrine—the most numerous type that are found all over the body, particularly on the palms of the hands, soles of the feet and forehead;

Apocrine—mostly confined to the armpits (axilla) and the anal-genital area. They typically end in hair follicles rather than pores.

Sweat is produced in both sweat glands in the same way. Eccrine sweat glands are small, active from birth, and produce a sweat free of proteins and fatty acids. However, the sweat from apocrine glands contains proteins and fatty acids, which make it thicker and give it a milkier or yellowish color. This is why underarm stains in clothing appear yellowish. When bacteria on the skin and hair metabolize the proteins and fatty acids, they produce an unpleasant odor. This is why deodorants and antiperspirants are applied to the underarms instead of the whole body. A recent study has shown that although there are quite a few resident populations that contribute to axillary odor, only the Corynebacterium have been shown to have a direct association between bacterial population and malodor intensity. Two main compounds are significant contributors to underarm odor:

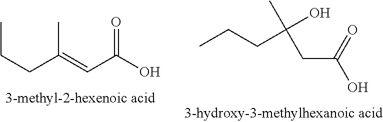

Biocontrol of the organisms producing the volatiles, prevention or reduction of conversion of precursor molecules to such volatiles or the prevention of secretion of such compounds by organisms, or consumption of the volatiles themselves before volatilization are desirable for control of underarm odor. In particular, strains that show zones of inhibition (ZOIs) against the key malodor causing bacteria such as Corynebacterium and Staphylococcus can be important.

### SUMMARY

The present invention provides, in one aspect, a method of inhibiting production of body malodormalodor caused by bacteria capable of causing body malodor by contacting the malodor-causing bacteria with at least one species of Bacillus or a substance derived from therefrom, where the at least one species of Bacillus is selected from the group consisting of Bacillus subtilis, Bacillus amyloliquefaciens, Bacillus pumilus, Bacillus licheniformis, Bacillus megaterium, Bacillus atrophaeus, and Bacillus mojavensis.

In a preferred embodiment, the species of Bacillus is Bacillus pumilus.

In a preferred embodiment, the bacteria causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.

In one aspect, the invention provides a method of inhibiting production of malodor caused by bacteria capable of causing malodor by contacting the bacteria with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain -NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642. More preferably, the at least one strain of Bacillus is selected from the group consisting of Bacillus pumilus strain NRRL B-50016; Bacillus amyloliquefaciens strain NRRL B-50018; Bacillus amyloliquefaciens strain PTA-7543; Bacillus amyloliquefaciens strain PTA-7544; Bacillus amyloliquefaciens strain PTA-7549; Bacillus subtilis strain NRRL B-50136; Bacillus amyloliquefaciens strain NRRL B-50304; Bacillus amyloliquefaciens strain PTA-7790; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus pumilus strain NRRL B-50398; Bacillus pumilus strain NRRL B-50396; and Bacillus pumilus strain NRRL B-50397. Most preferably, the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus pumilus strain NRRL B-50396; and Bacillus pumilus strain NRRL B-50397.

In a preferred embodiment, the bacteria causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.

In one aspect, the invention provides a method of inhibiting production of body malodor caused by Corynebacterium mucifaciens by contacting the Corynebacterium mucifaciens with at least one strain of Bacillus or a substance derived from therefrom, wherein the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain NRRL B-50018; Bacillus amyloliquefaciens strain PTA-7544; Bacillus amyloliquefaciens strain PTA-7549; Bacillus amyloliquefaciens strain PTA-7790; and Bacillus amyloliquefaciens strain NRRL B-50399. More preferably, the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain NRRL B-50018; Bacillus amyloliquefaciens strain PTA-7549; and Bacillus amyloliquefaciens strain PTA-7790. Most preferably, the at least one strain of Bacillus is Bacillus pumilus strain NRRL B-50016.

In one aspect, the invention provides a method of inhibiting production of body malodor caused by Corynebacterium diphtheriae by contacting the Corynebacterium diphtheriae with at least one strain of Bacillus or a substance derived from therefrom, wherein the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain PTA-7542; Bacillus amyloliquefaciens strain PTA-7544; Bacillus amyloliquefaciens strain PTA-7549; Bacillus amyloliquefaciens strain PTA-7790; and Bacillus subtilis strain NRRL B-50136. More preferably, the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain PTA-7544; Bacillus amyloliquefaciens strain PTA-7549; and Bacillus subtilis strain NRRL B-50136. Most preferably, the at least one strain of Bacillus is selected from the group consisting of Bacillus pumilus strain NRRL B-50016; Bacillus amyloliquefaciens strain PTA-7549; Bacillus pumilus strain NRRL B-50514; and Bacillus pumilus strain NRRL B-50515

In one aspect, the invention provides a method of inhibiting production of body malodor caused by Corynebacterium xerosis by contacting the Corynebacterium xerosis with at least one strain of Bacillus or a substance derived from therefrom, wherein the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; and Bacillus amyloliquefaciens strain NRRL B-50399. More preferably, the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7543; Bacillus amyloliquefaciens strain PTA-7545; Bacillus amyloliquefaciens strain PTA-7546; Bacillus amyloliquefaciens strain PTA-7549; Bacillus amyloliquefaciens strain PTA-7790; Bacillus amyloliquefaciens strain PTA-7791; and Bacillus amyloliquefaciens strain NRRL B-50304. Most preferably, the at least one strain of Bacillus is selected from the group consisting of Bacillus pumilus strain NRRL B-50016; Bacillus pumilus strain NRRL B-59652; Bacillus amyloliquefaciens strain NRRL B-50018; and Bacillus amyloliquefaciens strain NRRL B-50304.

In one aspect, the invention provides a method of inhibiting production of body malodor caused by Staphylococcus epidermidis by contacting the Staphylococcus epidermidis with at least one strain of Bacillus or a substance derived from therefrom, wherein the at least one strain of Bacillus is selected from the group consisting of Bacillus pumilus strain NRRL B-50016; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7793; Bacillus subtilis strain NRRL B-50136; Bacillus amyloliquefaciens strain NRRL B-50304; and Bacillus amyloliquefaciens strain NRRL B-50399. More preferably, the at least one strain of Bacillus is selected from the group consisting of Bacillus pumilus strain NRRL B-50016; Bacillus amyloliquefaciens strain PTA-7541; Bacillus amyloliquefaciens strain PTA-7543; Bacillus amyloliquefaciens strain PTA-7544; Bacillus amyloliquefaciens strain PTA-7793; Bacillus subtilis strain NRRL B-50136; Bacillus amyloliquefaciens strain NRRL B-50304; Bacillus pumilus strain NRRL B-50514; Bacillus pumilus strain NRRL B-50515; Bacillus pumilus strain NRRL B-59651; Bacillus pumilus strain NRRL B-59652; Bacillus amyloliquefaciens strain NRRL B-59650; and Bacillus amyloliquefaciens strain NRRL B-50399. Most preferably, the at least one strain of Bacillus is selected from the group consisting of Bacillus pumilus strain NRRL B-50016; Bacillus amyloliquefaciens strain PTA-7543; Bacillus pumilus strain NRRL B-50514; Bacillus pumilus strain NRRL B-50515; Bacillus pumilus strain NRRL B-59651; Bacillus pumilus strain NRRL B-59652; and Bacillus amyloliquefaciens strain NRRL B-59650.

In one aspect, the invention provides a method of inhibiting production of body malodor caused by Brevibacterium epidermidis by contacting the Brevibacterium epidermidis with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; and Bacillus amyloliquefaciens strain NRRL B-50399. More preferably, the at least one strain of Bacillus is selected from the group consisting of Bacillus pumilus strain NRRL B-50016; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7543; Bacillus amyloliquefaciens strain PTA-7544; Bacillus amyloliquefaciens strain PTA-7545; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus amyloliquefaciens strain NRRL B-50304; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; and Bacillus amyloliquefaciens strain NRRL B-50399. Most preferably, the at least one strain of Bacillus is selected from the group consisting of Bacillus pumilus strain NRRL B-50016; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquefaciens strain PTA-7543; Bacillus amyloliquefaciens strain PTA-7790; and Bacillus amyloliquefaciens strain NRRL B-50304.

In one aspect, the invention provides a composition adapted for application to the skin of a human comprising at least one species of Bacillus or a substance derived from therefrom, wherein the at least one species of Bacillus is selected from the group consisting of selected from the group consisting of Bacillus subtilis, Bacillus amyloliquefaciens, and Bacillus pumilus.

Most preferably, the species of Bacillus is Bacillus pumilus.

In one embodiment, the composition is a spray or powder. In one embodiment, the composition is solid or gel composition adapted for application as a deodorant.

In one embodiment, the at least one species of Bacillus is a strain of Bacillus selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus pumilus strain NRRL B-50396; and Bacillus pumilus strain NRRL B-50397. More preferably, the at least one strain of Bacillus is selected from the group consisting of Bacillus pumilus strain NRRL B-50016; Bacillus amyloliquefaciens strain NRRL B-50018; Bacillus amyloliquefaciens strain PTA-7543; Bacillus amyloliquefaciens strain PTA-7544; Bacillus amyloliquefaciens strain PTA-7549; Bacillus subtilis strain NRRL B-50136; Bacillus amyloliquefaciens strain NRRL B-50304; Bacillus amyloliquefaciens strain PTA-7790; Bacillus pumilus strain NRRL B-50398; Bacillus pumilus strain NRRL B-50396; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; and Bacillus pumilus strain NRRL B-50397. Most preferably, the at least one strain of Bacillus is selected from the group consisting of Bacillus pumilus strain NRRL B-50016; Bacillus pumilus strain NRRL B-50396; and Bacillus pumilus strain NRRL B-50397.

In one aspect, the invention provides a composition, adapted for application to the feet of a human, comprising at least one species of Bacillus or a substance derived from therefrom, wherein the at least one species of Bacillus is selected from the group consisting of selected from the group consisting of Bacillus subtilis, Bacillus amyloliquefaciens, and Bacillus pumilus. More preferably, the at least one species of Bacillus is a Bacillus strain selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain-NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59646; Bacillus subtilis strain NRRL B-59648; Bacillus subtilis strain NRRL B-59654; and Bacillus subtilis strain NRRL B-59642; and Bacillus amyloliquefaciens strain NRRL B-50399. More preferably, the at least one strain of Bacillus is selected from the group consisting of Bacillus pumilus strain NRRL B-50016; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7543; Bacillus amyloliquefaciens strain PTA-7544; Bacillus amyloliquefaciens strain PTA-7545; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus amyloliquefaciens strain NRRL B-50304; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; and Bacillus amyloliquefaciens strain NRRL B-50399. Most preferably, the at least one strain of Bacillus is selected from the group consisting of Bacillus pumilus strain NRRL B-50016; Bacillus amyloliquefaciens strain PTA-7543; Bacillus amyloliquefaciens strain PTA-7790; and Bacillus amyloliquefaciens strain NRRL B-50304.

In one aspect, the invention provides a method of inhibiting production of body malodor caused by Brevibacterium epidermidis by contacting the Brevibacterium epidermidis with Bacillus pumilus. More preferably, the Bacillus pumilus is at least one strain selected from the group consisting of Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; and Bacillus pumilus strain NRRL B-50397. Most preferably, the Bacillus pumilus is Bacillus pumilus strain NRRL B-50397.

In one aspect, the method comprises contacting the bacteria capable of causing body malodor. In another aspect, the method comprises contacting an odor generating compound derived from the bacteria capable of causing odor. In yet another aspect, the contacting comprises administering at least one beneficial microorganism to skin, but is not limited to skin.

In another aspect, the invention relates to strains that are closely related to each other on the basis of 16S rDNA sequence identity. In a preferred embodiment, a culture of the invention is preferably greater than 95% identical, more preferably greater than 97% identical, most preferably greater than 98.5% identical to species or strains specifically identified herein.

Combinations of aspects and embodiments form further embodiments of the present invention.

### BRIEF DESCRIPTION OF THE DRAWINGS

FIG. 1 is an overlay of GC-MS chromatograghs of volatile compounds in ASS medium grown with (shown in pink) or without (shown in black) S. epidermidis (ATCC 14990).

### DETAILED DESCRIPTION

Bacilli generating zones of inhibition (ZOIs) against key malodor causing bacteria above about 3 mm can be effective biocontrol agents in accordance with the present invention, and those generating ZOIs above about 6 mm are preferred. Therefore, the present invention includes testing bacilli for inhibition of Corynebacterium, Brevibacterium, and Staphylococcus strains. Also, the effect of these strains on production of the volatiles, prevention or reduction in conversion of precursor to such volatiles, or consumption of the volatiles can indicate usefulness in the compositions and methods of the invention.

As used herein, “inhibiting malodor production” means reducing or substantially eliminating malodor caused by odor-causing bacteria commonly associated with the production of body odor in humans or animals, preferably humans. Reducing or substantially eliminating the odor may occur by one or more effects associated with the bacterial control species and strains of the invention. These effects include, but are not limited to, inhibition of growth of the bacterial species, inhibition of the production or secretion of odorous volatile substances by odor-causing bacteria, by inhibition of the conversion of a chemical precursor into an odorous substance, or by modification of the odorous substance, each upon contact with the control species or a substance derived therefrom.

A biocontrol agent of the invention is a species or strain of Bacillus or a substance derived therefrom, that has action against the odor-causing organism or the secretion or production of an odor-causing chemical (e.g., volatile substance) in such manner as to reduce or eliminate the perceived odor arising from such organisms and volatiles, or degredation of the odor-causing molecule after secretion by the odor-causing organism. The methods and compositions of the invention include strains that are closely related to each other on the basis of 16S rDNA sequence identity. A culture useful according to the invention is preferably greater than 95% identical, more preferably greater than 97% identical, most preferably greater than 98.5% identical to species or strains specifically identified herein. See Stackebrandt E, et al., “Report of the ad hoc committee for the re-evaluation of the species definition in bacteriology,” Int J Syst Evol Microbiol. 52(3):1043-7 (2002) regarding use of 16S rDNA sequence identity for determining relatedness in bacteria.

Contacting an odor-causing organism with a biocontrol agent of the invention means contacting the organism with living cells of the control species, e.g., spores or vegetative cells, or contacting the odor-causing organism with a substance derived from the biocontrol agent. Such substances include, but are not limited to, cell-free supernatants, cell lysates, extracts, and the like.

The biocontrol bacteria of the invention, or substances derived therefrom can be used to prepare personal care products such as deodorants, including, in particular, preparations for underarm use on humans, and sprays, powders, solids, creams, etc., for use on humans or animals. Preparations for application to reduce or eliminate foot-odor are also included. Guidance regarding preparation and use of compositions for control of foot-odor can be found in published US patent application, Publication Number US 2009/0130073 A1 entitled “Microorganisms Inhibiting the Formation of Foot Malodor,” incorporated herein by reference for information relating to such compositions and their use.

For deodorants used for control of axillary malodor, formulations are provided. Live biocontrol bacterial, e.g., spores or vegetative cells, can be used. In addition to the examples provided herein, guidance for additional methods of evaluation of Bacillus species and strains useful according to the present invention, as well as guidance for production of compositions of the invention, can be found in published U.S. patent application, Publication No. US 2008/0247993 A1 entitled “Microorganisms inhibiting the formation of axillary malodor,” incorporated herein by reference for information relating to such methods and compositions. In particular, methods of evaluating suppression of volatile fatty acids and their odorous derivatives can be used. Also, methods involving both live and inactivated, e.g., in accordance with the present invention, spores and vegetative cells of Bacillus, can be useful. Further, the axillary secretions can be used as the source of odorless precursor compounds in accordance with the methods of present invention, in addition to use of artificial sweat medium containing short and medium chain fatty acids, rather than collected human axillary sweat. Further, an in vitro assay for determining blocking release of odor causing compounds is provided and can be used in the methods of the present invention. US 2008/0247993 A1 also provides specific information regarding additional odor-causing organisms that can be used in methods of evaluating control species according to the present invention, e.g., Corynebacterium bovis, Corynebacterium jeikelum (e.g., DSM 7171), or Corynebacterium striatum.

Regarding compositions and use of US 2008/0247993 A1, cell fractions or supernatants can provide active components for the methods and compositions of the present invention. In addition to spores or vegetative cells, cell-free supernatants and cell lysates can be used. Products, compositions, carriers, etc., as disclosed in US 2008/0247993 A1 can be useful according to the present invention. US 2008/0247993 A1 provides formulation examples for balms, gels, sticks, liquids, shampoos, etc., which can be useful according to the present invention, taking into account the foregoing regarding preparations incorporating live Bacillus. Relevant methods and compositions include impregnation in textiles, and odor reduction in textiles. However, for embodiments comprising live spores or vegetative cells of Bacillus, cationic surfactants and preservatives should generally be avoided.

For preservation of products comprising Bacillus sp., the following preservatives can be useful: chloromethylisothiazolinone/methylisothiazolinone (CMIT/MIT) (Kathon or others); MIT (Neolone or others); 1,2-benzisothiazolin-3-one (BIT) (if allowed in personal care); CMIT/MIT+EDTA; CMIT/MIT+Biodegradable Chelator; MIT+EDTA; MIT+Biodegradable Chelator; BIT+EDTA; BIT+Biodegradable Chelator; Bronopol; 2-Phenoxyethanol; 2-Phenoxyethanol+Biodegradable Chelator; Potassium sorbate (used at low pH); Sodium benzoate (used at low pH); Salt; Glycerol; Propylene Glycol; Essential Oils; Dichlorobenzyl alcohol; Triclosan; Parabens; and 1-Phenoxy-2-propanol and 2-Phenoxy-1-propanol. More preferably, the preservative is 2-Phenoxyethanol; 2-Phenoxyethanol+Biodegradable Chelator; Potassium Sorbate (used at low pH); Sodium Benzoate (used at low pH); Salt; Glycerol; Propylene Glycol; or one of more Essential Oils—e.g., white mustard seed, tea tree, rosewood, or some citrus oils. Most preferably, the preservative is 2-Phenoxyethanol; 2-Phenoxyethanol+Biodegradable Chelator; or Glycerol.

Essential oils useful according to the present invention include, but are not limited to, Rosewood, Celery seed, Frankincense, Ylang ylang, Cedarwood, Lime, Orange, Petitgrain, Bergamot, Lemon, Grapefruit, Mandarin, Myrrh, Coriander, Pumpkin, Cypress, Lemongrass, Palmarosa, Citronella, Carrot seed, Eucalyptus, Fennel, Wintergreen, Juniper, French lavender, Tasmanian lavender, Macadamia, Tea tree, Cajuput, Niaouli, Peppermint, Spearmint, Basil, Evening primrose, Marjoram, Oregano, Geranium, Aniseed, Bay, Pine, Black pepper, Patchouli, Apricot kernel, Sweet almond, Rosemary, Sage, Clary sage, Sandalwood, Clove, Thyme, Vetiver, and Ginger. Additional guidance regarding selection of appropriate essential oils may be found in Hammer, K. A., et al., J. Applied Microbiol., 86:985-990 (1999), incorporated herein by reference for its disclosure of essential oils/plant extracts and their antimicrobial activity.

The Bacillus strains used in the experiments herein are known as biocontrol strains. The following is a list of all of the recognized biocontrol strains. The Bacillus strains were maintained and cultivated on Standard Method Agar (SMA) for plate cultures and Plate Count Broth (PCB) for liquid cultures.

TABLE 1
Bacillus Strains and Deposit Information

| Identification | Accession Number | Date of Deposit |
|---|---|---|
| Bacillus licheniformis | NRRL B-50014* | 14 Mar. 07 |
| Bacillus licheniformis | NRRL B-50015 | 14 Mar. 07 |
| Bacillus pumilus | NRRL B-50016 | 14 Mar. 07 |
| Bacillus amyloliquifaciens | NRRL B-50017 | 14 Mar. 07 |
| Bacillus amyloliquifaciens | NRRL B-50018 | 14 Mar. 07 |
| Bacillus amyloliquifaciens | PTA-7541** | 20 Apr. 06 |
| Bacillus amyloliquifaciens | PTA-7792 | 18 Aug. 06 |
| Bacillus amyloliquifaciens | PTA-7542 | 20 Apr. 06 |
| Bacillus amyloliquifaciens | PTA-7543 | 20 Apr. 06 |
| Bacillus amyloliquifaciens | PTA-7544 | 20 Apr. 06 |
| Bacillus amyloliquifaciens | PTA-7545 | 20 Apr. 06 |
| Bacillus amyloliquifaciens | PTA-7546 | 20 Apr. 06 |
| Bacillus subtilis subsp. subtilis | PTA-7547 | 20 Apr. 06 |
| Bacillus amyloliquifaciens | PTA-7549 | 20 Apr. 06 |
| Bacillus amyloliquifaciens | PTA-7793 | 18 Aug. 06 |
| Bacillus amyloliquifaciens | PTA-7790 | 18 Aug. 06 |
| Bacillus amyloliquifaciens | PTA-7791 | 18 Aug. 06 |
| Bacillus subtilis subsp. subtilis | NRRL B-50136 | 30 May 10 |
| Bacillus amyloliquifaciens | NRRL B-50304 | 19 Jul. 09 |
| Bacillus amyloliquifaciens | NRRL B-50399 | 21 Jun. 10 |
| Bacillus pumilus | NRRL B-50398 | 21 Jun. 10 |
| Bacillus pumilus | NRRL B-59643 | 15 Jun. 11 |
| Bacillus pumilus | NRRL B-59644 | 15 Jun. 11 |
| Bacillus pumilus | NRRL B-59645 | 15 Jun. 11 |
| Bacillus pumilus | NRRL B-50396 | 21 Jun. 10 |
| Bacillus pumilus | NRRL B-50397 | 21 Jun. 10 |
| Bacillus pumilus | NRRL B-50255 | 19 Feb. 09 |
| Bacillus licheniformis | NRRL B-1001 | 19 Jul. 49 |
| Bacillus megaterium | NRRL B-14308 | 30 Aug. 85 |
| Bacillus megaterium | PTA-3142 | 01 Mar. 01 |
| Bacillus pumilus | NRRL B-59658 | 22 Jun. 11 |
| Bacillus mojavensis | NRRL B-59636 | 20 May 11 |
| Bacillus mojavensis | NRRL B-59656 | 15 Jun. 11 |
| Bacillus pumilus | NRRL B-50514 | 20 May 11 |
| Bacillus pumilus | NRRL B-50515 | 20 May 11 |
| Bacillus pumilus | NRRL B-59651 | 15 Jun. 11 |
| Bacillus pumilus | NRRL B-59652 | 15 Jun. 11 |
| Bacillus pumilus | NRRL B-59655 | 15 Jun. 11 |
| Bacillus amyloliquifaciens | NRRL B-59657 | 15 Jun. 11 |
| Bacillus amyloliquifaciens | NRRL B-59647 | 15 Jun. 11 |
| Bacillus amyloliquifaciens | NRRL B-59649 | 15 Jun. 11 |
| Bacillus amyloliquifaciens | NRRL B-59650 | 15 Jun. 11 |
| Bacillus amyloliquifaciens | NRRL B-59653 | 15 Jun. 11 |
| Bacillus subtilis | NRRL B-59646 | 15 Jun. 11 |
| Bacillus subtilis | NRRL B-59648 | 15 Jun. 11 |
| Bacillus subtilis | NRRL B-59654 | 15 Jun. 11 |
| Bacillus subtilis | NRRL B-59642 | 15 Jun. 11 |

*NRRL indicates deposit with the Agricultural Research Service Culture Collection, Peoria, IL
**PTA indicates deposit with the American Type Culture Collection

The following examples are included for illustrative purposes only and are not intended to limit the scope of the invention.

### EXAMPLES

### Example 1

### Initial Zone of Inhibition Experiments

### Introduction

The present invention arose from an effort to produce a product that would inhibit growth of Corynebacterium in the human axilla with effectiveness in a deodorant or other personal care composition. In Taylor, D., et al., “Characterization of the microflora of the human axilla,” International Journal of Cosmetic Science, 25:137-145 (2003), aerobic coryneforms were shown to have a ρ value of <0.0001 when population counts were correlated to malodor intensity. Another article, James, A. G., et al., “Fatty acid metabolism by cutaneous bacteria and its role in axillary malodor,” World Journal of Microbiology and Biotechnology, 20:787-793 (2004), proposed the metabolic pathways used by aerobic coryneforms that would generate malodor.

### Methods

### Media and Strains

Based on demonstrated relevance to the origin of human axillary malodor, the ATCC was contacted and three strains were ordered: Corynebacterium mucifaciens (ATCC 700355) and Corynebacterium diphtheriae (ATCC 11913). C. mucifaciens and C. diphtheriae were cultivated without incident in 7 mL of Tryptic Soy Broth (TSB) with 1 mL of Tween 80 per liter. The strains were allowed to incubate overnight at 35° C. and were then struck out onto both a beef extract based agar and Tryptic Soy Agar (TSA) with 0.1% Tween 80 where the components are described below:

TABLE 2
Media
Media

| Beef Extract Media (g/L) | | TSB-w/0.1% Tween 80 (g/L) | | | TSA w/0.1% Tween 80 (g/L) | | |
|---|---|---|---|---|---|---|---|
| Agar | 15.0 g | Pancreatic Digest | 17.0 | g | Pancreatic Digest | 17.0 | g |
| | | of Casein | | | of Casein | | |
| Beef Extract | 10.0 g | Papaic Digest | 3.0 | g | Papaic Digest | 3.0 | g |
| | | of Soybean | | | of Soybean | | |
| Peptone | 10.0 g | Dextrose | 2.5 | g | Dextrose | 2.5 | g |
| NaCl | 5.0 g | NaCl | 5.0 | g | NaCl | 5.0 | g |
| | | Dipotassium | 2.5 | g | Dipotassium | 2.5 | g |
| | | phosphate | | | phosphate | | |
| | | Tween 80 | 1 | mL | Tween 80 | 1 | mL |
| | | | | | Agar | 15.0 | g |

The Beef Extract Media composition was prepared in accordance with page 373 of the Handbook of Microbiological Media under the title of “Corynebacterium Agar.” The TSA and TSB-medias with 0.1% Tween were acquired from the ATCC website as optimal media in which to culture the strains in both liquid and plate cultures. The TSA and TSB-media used in this experiment were premixed by Bacto Laboratories, Pty, Ltd(BD*BBL Tryptic Soy Broth (Soybean-Casein Digest Medium) (211825), BD*BBL/Difco Granulated Agar (214510), and Tween80, Fisher BioReagents (Bp338-500), available from Fisher Scientific; Beef Extract=Beef Extract Powder (supplied by VWR-61001-510) & Bacto Peptone, Fine Powder (211677) & NaCl (Crystalline/Biological, Certified-S671).

### Example 2

### Experimental Design and Implementation

The overall experimental design for the experiments of this example is as follows. Overnight cultures of the Bacillus and Corynebacterium strains were grown at 35° C. overnight in 7 mL of media where the Bacillus strains were cultured in PCB and the Corynebacterium strains were cultivated in TSB with 0.1% Tween 80 and where one colony from a reference plate was the seed inoculum for the culture tube.

The following day a 100 mL aliquot of 0.75% agar solution was created and autoclaved, as well as 6 mm sterile paper disks in a glass Petri dish and a container of 1.5 mL centrifuge tubes. A water bath was placed in the BSL-2 hood and set for 47° C. During the autoclave run the plates were removed from the cold room and placed in the BSL-2 hood in order to reach room temperature. Once the autoclave had finished its run, the soft agar solution was placed in the water bath and allowed to cool for approximately 1 hour. After the soft agar had appropriately cooled, 4 mL were aliquoted into sterile 15 mL falcon tubes. After enough tubes were created the rack of falcon tubes was placed back in the water bath to ensure that the soft agar would not solidify before the plates were ready.

Next, the Corynebacterium samples were removed from the incubator and 1 mL of the overnight culture was transferred to a sterile 1.5 mL centrifuge tube.

Next 100 μL of the Corynebacterium sample was added to 4 mL of the soft agar. The tube was capped, briefly vortexed, and then soft agar/bacteria mixture was poured onto the plate. To ensure even dispersal of the soft agar, the plate was gently swirled until the entire surface area of the plate was covered. The plate was then moved to the back of the hood where it could cool without disturbance. Using this method an evenly dispersed bacterial lawn was created. The process was repeated for as many plates as necessary ensuring that only one Corynebacterium strain is applied to any individual plate.

Once all of the soft agar plates were made and the soft agar had set, the overnight Bacillus cultures were removed from the shaker and placed in a laminar flow hood and 1 mL of the individual Bacillus strains was aliquoted into sterile 1.5 mL centrifuge tubes. The soft agar plates were then brought over to the hood and using a sterile needle the 6 mm sterile paper disks were applied on top of the soft agar. After writing the strain number on the back of the plate and applying the disk to the soft agar, 10 uL of overnight Bacillus culture was pipetted onto the disk. Once finished, the plates were incubated at 35° C. overnight.

The next day, the plates were removed and examined for Zones of Clearing/Inhibition. If there was no observable zone around the paper disk or the colony, then a “_” was recorded, if there was a zone around the disk or colony, then:

the distance from the colony's edge to the zone was recorded;

the diameter of the zone was recorded; and

the diameter of the colony was recorded.

### Beef Extract Plates and Tween 80 Plates

The table shown below was the only data acquired in the breach of the method mentioned above. Instead of 4 mL of soft agar overlay only 1 mL was used and the overlay was spread around the plate using a spreader. The experiment also used both Beef Extract based plates and TSB-with 0.1% Tween 80 plates in order to see if the plate media would affect inhibition.

TABLE 3
C. mucifaciens and C. diphtheriae results - Beef Extract and Tween 80 Plates

| | C. mucifaciens (ATCC 700355) | | | | C. diphtheriae (ATCC 11913) | | | |
|---|---|---|---|---|---|---|---|---|
| | Edge of Colony to | | Ratio of Colony | | Edge of Colony to | | Ratio of Colony | |
| | Edge of Zone (mm) | | Size to Zone Size | | Edge of Zone (mm) | | Size to Zone Size | |
| Access. | Beef Extr. | TSA w/0.1% | Beef Extr. | TSA w/0.1% | Beef Extr. | TSA w/0.1% | Beef Extr. | TSA w/0.1% |
| No. | Media* | DET** | Media | DET | Media | DET | Media | DET |
| NRRL B-50014 | — | — | — | — | — | — | — | — |
| NRRL B-50015 | — | — | — | — | — | — | — | — |
| NRRL B-50016 | Partial 5 | — | Smear | — | — | — | — | — |
| NRRL B-50017 | — | — | — | — | — | — | — | — |
| NRRL B-50018 | Partial 2 | Partial 3 | 1;1 | 1;2 | — | — | — | — |
| PTA-7542 | — | Partial 2 | — | 1;1.8 | 1 | — | 1;1.15 | — |
| PTA-7544 | 3 | Partial 1 | 1;1.4 | 1;1.2 | Partial 2 | Partial 2 | 1;1.25 | 1;2 |
| PTA-7546 | — | 3 | — | 1;2.3 | — | — | — | — |
| PTA-7549 | 4 | Partial 1 | 1;2.5 | — | 3 | — | 1;1.42 | — |
| PTA-7790 | 3 | 5 | 1;1.7 | Smear | 1 | — | 1;1.33 | — |
| NRRL B-50136 | 2 | Partial 3 | 1;1.7 | 1;2.5 | 2 | — | 1;2 | — |

*Beef Extract Media;
**Tween 80

The results indicated that the beef extract media increases the ability of the Bacillus strains to inhibit the Corynebacterium lawns. So the Beef Extract Plates were used exclusively from this experiment onward.

### Beef Extract Plate Repeat Data

The experiment was repeated using exclusively Beef Extract based plates except this time 4 mL of soft agar was poured onto the plate and then spread via spreader. The results are shown below and the experiment was performed in duplicate. Under the Plate ID column the muc prefix indicates that the lawn was C. mucifaciens and the dip prefix indicates a lawn of C. diphtheriae.

TABLE 4
Beef Extract Results
Experimental Data

| | | Edge of | | | |
|---|---|---|---|---|---|
| | | Col to | | | |
| | | Edge of | Diameter | Diameter | Zone/ |
| Plate | Access. | Zone | of Colony | of Zone | Colony |
| ID | No. | (mm) | (mm) | (mm) | Ratio |
| muc 1 | NRRL B-50015 | — | — | — | — |
| muc 1 | NRRL B-50014 | — | — | — | — |
| muc 1 | PTA-7542 | 2.00 | 15.00 | 20.00 | 1:1.33 |
| muc 1 | PTA-7544 | 2.00 | 19.00 | 20.00 | 1:1.05 |
| muc 2 | NRRL B-50136 | 1.00 | 10.00 | 13.00 | 1:1.30 |
| muc 2 | NRRL B-50018 | 1.00 | 18.00 | 21.00 | 1:1.17 |
| muc 2 | PTA-7546 | 2.00 | 15.00 | 17.00 | 1:1.13 |
| muc 3 | NRRL B-50136 | 2.00 | 11.00 | 14.00 | 1:1.27 |
| muc 3 | NRRL B-50018 | 2.00 | 15.00 | 20.00 | 1:1.33 |
| muc 3 | PTA-7546 | — | — | — | — |
| muc 4 | PTA-7549 | 3.00 | 15.00 | 18.00 | 1:1.20 |
| muc 4 | NRRL B-50016 | 2.00 | SMEAR | SMEAR | — |
| muc 4 | NRRL B-50017 | — | — | — | — |
| muc 4 | PTA-7790 | 2.00 | 18.00 | 21.00 | 1:1.17 |
| muc 5 | PTA-7549 | 2.00 | 14.00 | 18.00 | 1:1.29 |
| muc 5 | NRRL B-50016 | — | — | — | — |
| muc 5 | NRRL B-50017 | 3.00 | 6.00 | 10.00 | 1:1.67 |
| muc 5 | PTA-7790 | 3.00 | 13.00 | 18.00 | 1:1.38 |
| muc 6 | NRRL B-50015 | — | — | — | — |
| muc 6 | PTA-7544 | 1.00 | 19.00 | 20.00 | 1:1.05 |
| muc 6 | NRRL B-50014 | — | — | — | — |
| muc 6 | PTA-7542 | 2.00 | 15.00 | 20.00 | 1:1.33 |
| dip 1 | NRRL B-50136 | 3.00 | 12.00 | 16.00 | 1:1.33 |
| dip 1 | NRRL B-50018 | — | — | — | — |
| dip 1 | PTA-7546 | — | — | — | — |
| dip 2 | PTA-7549 | 3.00 | 10.00 | 17.00 | 1:1.70 |
| dip 2 | NRRL B-50016 | — | — | — | — |
| dip 2 | NRRL B-50017 | — | — | — | — |
| dip 2 | PTA-7790 | — | — | — | — |
| dip 3 | NRRL B-50136 | 3.00 | 9.00 | 15.00 | 1:1.67 |
| dip 3 | NRRL B-50018 | — | — | — | — |
| dip 3 | PTA-7546 | — | — | — | — |
| dip 4 | NRRL B-50015 | — | — | — | — |
| dip 4 | PTA-7544 | 1.00 | 15.00 | 16.00 | 1:1.07 |
| dip 4 | NRRL B-50014 | — | — | — | — |
| dip 4 | PTA-7542 | 3.00 | 15.00 | 20.00 | 1:1.33 |
| dip 5 | PTA-7549 | 3.00 | 10.00 | 15.00 | 1:1.50 |
| dip 5 | NRRL B-50017 | — | — | — | — |
| dip 5 | NRRL B-50016 | — | — | — | — |
| dip 5 | PTA-7790 | 4.00 | 12.00 | 17.00 | 1:1.42 |
| dip 6 | NRRL B-50015 | — | — | — | — |
| dip 6 | PTA-7544 | 2.00 | 17.00 | 18.00 | 1:1.06 |

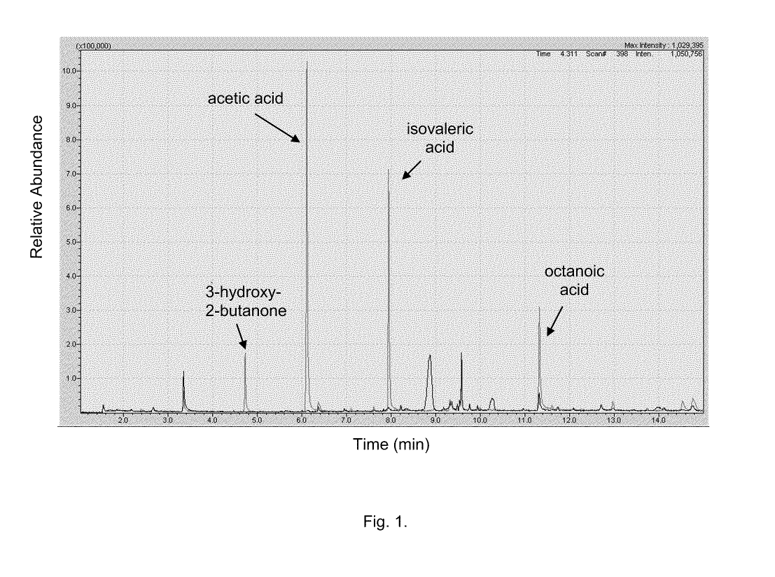

The image results for the C. mucifaciens screen are shown below in FIGS. 1A-1F. The image results for C. diphtheriae are shown in FIGS. 2A-2F.

### Third Experiment Using Beef Extract Plates

This experiment was conducted as described in the methods and yielded good presentable results. These data represent an experiment using C. diphtheriae.

TABLE 5
Further Beef Extract Results
Experimental Data

| | | Edge of | | | |
|---|---|---|---|---|---|
| | | Colony | | | |
| | | to Edge | Diameter | Diameter | Zone/ |
| Plate | Access. | of Zone | of Colony | of Zone | Colony |
| ID | No. | (mm) | (mm) | (mm) | Ratio |
| Dip 1 | NRRL B-50016 | — | — | — | — |
| Dip 1 | NRRL B-50014 | 1.00 | 14.00 | 16.00 | 1:1.14 |
| Dip 1 | PTA-7542 | 3.00 | 20.00 | 24.00 | 1:1.20 |
| Dip 2 | PTA-7546 | <1 | 23.00 | 24.00 | 1:1.04 |
| Dip 2 | PTA-7549 | 3.00 | 19.00 | 22.00 | 1:1.16 |
| Dip 2 | NRRL B-50136 | <1 | 17.00 | 18.00 | 1:1.06 |
| Dip 2 | NRRL B-50018 | <1 | 21.00 | 22.00 | 1:1.05 |
| Dip 3 | PTA-7544 | — | — | — | — |
| Dip 3 | NRRL B-50015 | — | — | — | — |
| Dip 3 | PTA-7790 | 4.00 | 16.00 | 24.00 | 1:1.50 |
| Dip 3 | NRRL B-50017 | — | — | — | — |

The images for C. diphtheriae in FIGS. 3A-3C correspond to the data above.

The results can be difficult to repeat with the exactitude that is expected with many other types of experiments. All conditions should be substantially identical in order to obtain similar results including: similar starting count for both Corynebacterium lawns and Bacillus sterile disks, incubation times need to be similar to ensure proper cell growth and/or metabolite production, dosing and incubation times need to be nearly identical as a few hours can make a large difference.

Additionally, it may be that smearing can be reduced if the sterile disks are inoculated prior to being placed on the plate, and it is recommended that a sterile microtiter plate is used as the holding vessel. The plates in the experiments herein were incubated face up allowing condensate to drip onto the plate and occasionally cause a smearing affect. The experiments were conducted knowing that condensate smearing was likely, however it was deemed necessary in order to ensure that the Bacillus inoculated disks did not fall from the plate during the overnight incubation period.

In additional experiments, Corynebacterium xerosis (ATCC Accession No. 373); Brevibacterium epidermidis (ATCC Accession No. 35514), and Staphylococcus epidermidis (ATCC Accession No. 14990) are considered as odor-causing species. Results are summarized further in the following Tables 6 and 7 and in FIGS. 4 and 5.

TABLE 6
ZOI Results on TSA with 0.1% Tween 80 Media

| Accession | C. | C. | C. | | | Average ZOI | Total of Zones |
|---|---|---|---|---|---|---|---|
| Number | MUC | DIP | XER | STAPH | BREVI | Across All Tests | Across All Medias |
| NRRL B-50136 | 0 | 0 | 0 | 0 | 0 | 0 | 12 |
| NRRL B-50015 | 0 | 0 | 0 | 0 | 0 | 0 | 0 |
| NRRL B-50016 | 7 | 3 | 7 | 2 | 6 | 6.25 | 42 |
| NRRL B-50017 | 1 | 0 | 2 | 0 | 1 | 1 | 7 |
| NRRL B-50018 | 2 | 0 | 3 | 0 | 2 | 1.75 | 16 |
| PTA-7541 | 1 | 0 | 3 | 0 | 2 | 1.5 | 14 |
| PTA-7792 | 1 | 0 | 3 | 0 | 1 | 1.25 | 10.5 |
| PTA-7542 | 1 | 0 | 1 | 0 | 1 | 0.75 | 6.5 |
| PTA-7543 | 1 | 0 | 2 | 0 | 2 | 1.25 | 20 |
| PTA-7544 | 1 | 0 | 3 | 0 | 1 | 1.25 | 16 |
| PTA-7545 | 1 | 0 | 3 | 0 | 1 | 1.25 | 12.5 |
| PTA-7546 | 2 | 0 | 3 | 0 | 1 | 1.5 | 8.5 |
| PTA-7547 | 1 | 0 | 2 | 0 | 1 | 1 | 10 |
| PTA-7549 | 1 | 0 | 3 | 0 | 2 | 1.5 | 15.5 |
| PTA-7793 | 1 | 0 | 2 | 0 | 1 | 1 | 12 |
| PTA-7790 | 2 | 0 | 3 | 0 | 2 | 1.75 | 18.5 |
| PTA-7791 | 1 | 0 | 3 | 0 | 1 | 1.25 | 11 |
| NRRL B-50304 | 1 | 0 | 4 | 0 | 3 | 2 | 19 |
| NRRL B-50399 | 2 | 0 | 3 | 0 | 2 | 1.75 | 12.5 |
| NRRL B-50398 | 2 | 2 | 4 | 2 | 4 | 2.8 | Not Tested on More |
| | | | | | | | than One Media |
| NRRL B-59643 | 2 | 0 | 3 | 0 | 4 | 1.8 | Not Tested on More |
| | | | | | | | than One Media |
| NRRL B-59644 | 2 | 0 | 7 | 1 | 4 | 2.8 | Not Tested on More |
| | | | | | | | than One Media |
| NRRL B-59645 | 0 | 0 | 1 | 0 | 0 | 0.2 | Not Tested on More |
| | | | | | | | than One Media |
| NRRL B-50396 | 4 | 3 | 9 | 0 | 5 | 4.2 | Not Tested on More |
| | | | | | | | than One Media |
| NRRL B-50397 | 3 | 4 | 8 | 1 | 7 | 4.6 | Not Tested on More |
| | | | | | | | than One Media |
| NRRL B-59657 | 1.8 | 1.1 | 2.9 | 0.0 | 1.6 | 1.5 | Not Tested on More |
| | | | | | | | than One Media |
| NRRL B-59646 | 2.3 | 1.1 | 1.6 | 0.3 | 1.3 | 1.3 | Not Tested on More |
| | | | | | | | than One Media |
| NRRL B-59647 | 1.6 | 1.5 | 3.1 | 0.0 | 2.0 | 1.6 | Not Tested on More |
| | | | | | | | than One Media |
| NRRL B-59648 | 1.4 | 0.8 | 3.0 | 0.0 | 0.6 | 1.2 | Not Tested on More |
| | | | | | | | than One Media |
| NRRL B-50514 | 5.0 | 4.5 | 4.8 | 2.6 | 3.8 | 4.1 | Not Tested on More |
| | | | | | | | than One Media |
| NRRL B-50515 | 5.5 | 4.8 | 4.0 | 3.2 | 3.6 | 4.2 | Not Tested on More |
| | | | | | | | than One Media |
| NRRL B-59649 | 1.4 | 0.0 | 1.6 | 0.2 | 1.5 | 0.9 | Not Tested on More |
| | | | | | | | than One Media |
| NRRL B-59650 | 2.8 | 1.3 | 3.1 | 0.0 | 3.3 | 2.1 | Not Tested on More |
| | | | | | | | than One Media |
| NRRL B-59651 | 3.3 | 1.8 | 3.2 | 2.9 | 4.3 | 3.1 | Not Tested on More |
| | | | | | | | than One Media |
| NRRL B-59652 | 5.5 | 2.8 | 6.5 | 2.8 | 4.1 | 4.3 | Not Tested on More |
| | | | | | | | than One Media |
| NRRL B-59653 | 1.2 | 0.1 | 3.8 | 0.2 | 2.5 | 1.5 | Not Tested on More |
| | | | | | | | than One Media |
| NRRL B-59654 | 1.9 | 2.1 | 1.3 | 1.3 | 0.8 | 1.5 | Not Tested on More |
| | | | | | | | than One Media |
| NRRL B-59655 | 6.5 | 2.5 | 3.5 | 2.8 | 1.3 | 3.3 | Not Tested on More |
| | | | | | | | than One Media |
| NRRL B-59656 | 2.3 | 1.5 | 1.0 | 1.1 | 1.3 | 1.5 | Not Tested on More |
| | | | | | | | than One Media |

*C. muc. = C. mucifaciens; C. dip. = C. diphtheriae; C. xer. = C. xerosis; Staph = Staphylococcus epidermidis; Brevi = Brevibacterium epidermidis.
All results are in mm.

TABLE 7
C. mucifaciens and C. diphtheriae on Beef Extract and TSA with Tween 80

| | | C. mucifaciens | | C. diphtheriae | |
|---|---|---|---|---|---|
| | | Edge of Colony to Edge of Zone | | Edge of Colony to Edge of Zone | |
| | | Beef Extract | TSA w/0.1% | Beef Extract | TSA w/0.1% |
| Accession No | Identification | Media | Tween 80 | Media | Tween 80 |
| NRRL B-50014 | Bacillus licheniformis | 0 | 0 | 0 | 0 |
| NRRL B-50015 | Bacillus licheniformis | 0 | 0 | 0 | 0 |
| NRRL B-50016 | Bacillus pumilus | 5 | 0 | 0 | 0 |
| NRRL B-50017 | Bacillus amyloliquifaciens | 0 | 0 | 0 | 0 |
| NRRL B-50018 | Bacillus amyloliquifaciens | 2 | 3 | 0 | 0 |
| PTA-7541 | Bacillus amyloliquifaciens | 0 | 0 | 0 | 0 |
| PTA-7792 | Bacillus atrophaeus | 0 | 0 | 0 | 0 |
| PTA-7542 | Bacillus amyloliquifaciens | 0 | 2 | 1 | 0 |
| PTA-7543 | Bacillus atrophaeus | 0 | 0 | 0 | 0 |
| PTA-7544 | Bacillus amyloliquifaciens | 3 | 3 | 2 | 2 |
| PTA-7545 | Bacillus amyloliquifaciens | 0 | 0 | 0 | 0 |
| PTA-7546 | Bacillus amyloliquifaciens | 0 | 3 | 0 | 0 |
| PTA-7547 | Bacillus subtilis | 0 | 0 | 0 | 0 |
| PTA-7549 | Bacillus amyloliquifaciens | 4 | 1 | 3 | 0 |
| PTA-7793 | Bacillus amyloliquifaciens | 0 | 0 | 0 | 0 |
| PTA-7790 | Bacillus amyloliquifaciens | 0 | 5 | 1 | 0 |
| PTA-7791 | Bacillus amyloliquifaciens | 0 | 0 | 0 | 0 |
| NRRL B-50136 | Bacillus subtilis | 2 | 3 | 2 | 0 |
| NRRL B-50304 | Bacillus amyloliquifaciens | Not Tested | 0 | Not Tested | 0 |
| NRRL B-50399 | Bacillus amyloliquifaciens | Not Tested | 0 | Not Tested | 0 |

TABLE 8
Average ZOI per Strain Across Different Media

| | Average ZOI per Strain Across Different Media | | | | |
|---|---|---|---|---|---|
| | C. | C. | C. | | |
| Accession No. | mucifaciens | diphtheriae | xerosis | Staph | Brevi |
| NRRL B-50014 | 0 | 0 | 0 | 0.0 | 0.0 |
| NRRL B-50015 | 0 | 0 | 0 | 0.0 | 0.0 |
| NRRL B-50016 | 6 | 1.5 | 4.5 | 2.2 | 3.8 |
| NRRL B-50017 | 0.5 | 0 | 2 | 0.0 | 0.7 |
| NRRL B-50018 | 2 | 0 | 3.5 | 0.0 | 1.7 |
| PTA-7541 | 0.5 | 0 | 2 | 0.7 | 2.3 |
| PTA-7792 | 0.5 | 0 | 2 | 0.3 | 1.5 |
| PTA-7542 | 0.5 | 0.5 | 1 | 0.2 | 0.7 |
| PTA-7543 | 0.5 | 0 | 2.5 | 1.7 | 3.0 |
| PTA-7544 | 2 | 1 | 1.5 | 0.7 | 1.7 |
| PTA-7545 | 0.5 | 0 | 2 | 0.3 | 2.2 |
| PTA-7546 | 1 | 0 | 2 | 0.0 | 0.8 |
| PTA-7547 | 0.5 | 0 | 1 | 0.0 | 2.3 |
| PTA-7549 | 2.5 | 1.5 | 2 | 0.0 | 1.2 |
| PTA-7793 | 0.5 | 0 | 1.5 | 0.7 | 2.0 |
| PTA-7790 | 2.5 | 0.5 | 2 | 0.0 | 2.8 |
| PTA-7791 | 0.5 | 0 | 2.5 | 0.0 | 1.7 |
| NRRL B-50136 | 1 | 1 | 0.75 | 0.8 | 1.3 |
| NRRL B-50304 | 0.5 | 0 | 3 | 0.7 | 3.3 |
| NRRL B-50399 | 1 | 0 | 1.5 | 0.7 | 1.8 |
| NRRL B-50398 | 2 | 2 | 4 | 2 | 4 |
| NRRL B-59643 | 2 | 0 | 3 | 0 | 4 |
| NRRL B-59644 | 2 | 0 | 7 | 1 | 4 |
| NRRL B-59645 | 0 | 0 | 1 | 0 | 0 |
| NRRL B-50396 | 4 | 3 | 9 | 0 | 5 |
| NRRL B-50397 | 3 | 4 | 8 | 1 | 7 |

### Example 3

### Gas Chromatograph-Mass Spectroscopy (GC-MS) Methodology Generation of Malodor Molecules by Odor-Causing Bacteria

Previous studies suggested that short-medium chain (C₂-C₁₁) volatile fatty acids (VFAs) are among the causal molecules of axillary malodor and foot malodor. In this experiment, we studied the odor metabolites produced by a proposed odor-causing bacterium Staphylococcus epidermidis (ATCC 14990).

Artificial sebum sweat (ASS) medium was made of Basal medium (750 mL/L), Sweat medium (230 mL/L), squalene (10 mL/L), and artificial sebum (10 mL/L). The Basal medium contains MgSO₄.7H₂O (0.5 g/L), KH₂PO₄ (1.0 g/L), CaCl₂ (1.11 mg/L), yeast extract (0.1 g/L), peptone (5.0 g/L), and glycerol (4.0 g/L); pH was adjusted to 7.5. The sweat medium contains NaCl (9.0 g/L), lactic acid (1.73 g/L), urea (1.07 g/L), casamino acids (0.20 g/L), NH₄Cl (0.18 g/L), creatinine (0.02 g/L), and uric acid (0.015 g/L); pH was adjusted to 7.0. The artificial human sebum contains w/w: 10% paraffin wax, 10% olive oil, 10% coconut oil, 25% cottonseed oil, 1.4% oleic acid, 5% palmitic acid, 1.2% cholesterol, and 37.4% water.

ATCC 14990 was inoculated into the ASS medium and ASS without bacterial culture was used as a control. After incubation at 35° C. for 72 to 96 hours, 1 mL of each culture was transferred to a GC headspace vial, and 20 μL of 3N HCl was added to each vial. The samples were then mixed briefly and analyzed with gas chromatography (GC-MS) using the method described below.

### GC-MS Method

A 50/30 μm divinylbenzene/Carboxen/polydimethylsiloxane (DVB/CAR/PDMS) solid phase micro-extraction (SPME) fiber (Supelco) is introduced into the headspace of vials (pre-equilibrated for 5 minutes at 50° C.) using the Combi Pal AOC 5000 autosampler (CTC Analytics). Extraction is carried out for 10 minutes at 80° C. Following extraction, the fiber is immediately introduced into a Shimadzu 2010-S gas chromatograph (GC) equipped with Siltek split/splitless inlet liner (Restek) and an Nukol fused silica capillary column (30 m×0.25 mm×0.25 μm film thickness; Sigma-Aldrich) connected to an electron impact quadropole mass spectrometer (MS) system. Injection port temperature is set to 200° C. The column is taken through the following program: 80° C. for 1 min, 15° C./min to 200° C., hold at 200° C. for 6 min. The total run time is 15 minutes. Two blank desorptions are performed prior to the first sample to free the fiber of analyte. The GC is operated with a split of 100 ml/min and purge of 0.5 ml/min. Grade 5 helium is used as the carrier gas (1 ml/min column flow). The MS ion source temperature is set to 180° C. Interface is set to 200° C. Scan mode is used (m/z 40-400). Peak areas are calculated with GC/MS solution software (Shimadzu). Compounds are identified by comparing their spectra to a standard library (NIST Mass Spectral Search Program).

The ASS medium (no bacteria control) had a very weak fatty smell before and after incubation. After incubation with ATCC 14990, the culture had developed an unpleasant acidic smell. Using GC/MS analysis, we compared the volatile compounds in cultures with or without ATCC 14990. The results are shown in FIG. 1. We identified four main peaks present in the culture with ATCC 14990 but absent in the control. These compounds are acetic acid, isovaleric acid, octanoic acid, and 3-hydroxy-2-butanone. Three out of the four compounds are short-medium chain fatty acids. These data suggest that odor-causing bacteria could convert artificial sweat/sebum to malodor molecules, mainly volatile fatty acids.

### Example 4

### Biodegradation of Malodor Molecules by Beneficial Bacteria

A biodegradation study was performed to determine whether NZB strains NRRL B-50014, NRRL B-50018, NRRL B-50255, NRRL B-50136, NRRL B-50015, NRRL-B59636, NRRL B-1001, PTA-7790, NRRL B-14308, PTA-3142, PTA-7549, PTA-7543, NRRL B-59658 and NRRL-B59642 could grow on and reduce odorous compounds known to be contributors to underarm malodor. Minimal medium (MM) was made as follows: Na₂HPO₄ (2.84 g/L), KH₂PO₄ (2.72 g/L), (NH₄)₂SO₄ (1 g/L), and Hunter's concentrated base (10 ml/L). Hunter's concentrated base was made as follows: EDTA (2.5 g/L), ZnSO₄.7H₂O (1.095 g/L), FeSO₄.7H₂O (698 mg/L), MnSO₄.H₂O (154 mg/L), CuSO₄.5H₂O (39.2 mg/L), Co(NO₃)₂.6H₂O (25 mg/L), Na₂B₄O₇.10H₂O (2.4 mg/L), nitrilotriacetic acid (20 g/L), KOH (14 g/L), MgSO₄ (28 g/L), CaCl₂.2H₂O (6.67 g/L), and (NH₄)6Mo₇O₂₄.2H₂O (18.5 mg/L). Volatile fatty acids (VFA) medium (per liter) was prepared by mixing 10 mL of a 10% glycerol, 60 mL of 10 mM VFA mixture (Sigma 46975-U), and 930 mL of MM medium. Bacillus strains were grown 18-24 hrs in MM with 10 mM glucose. 100 μL of each bacterial culture was transferred into 5 mL of VFA media. A tube of VFA medium without bacterial culture added was used as a control. All the cultures were incubated at 35° C. with shaking.

The amount of VFA in each culture was analyzed at 0 h, 24 h, 48 h, and 72 h incubation time. For each time point, 1 mL of culture was transferred to a GC headspace vial, and 20 μL of 3N HCl was added to each vial. The samples were then mixed briefly and analyzed with GC-MS using the method described above. Standard curves were generated by injection 0.05 mM, 0.1 mM, 0.2 mM, 0.3 mM, 0.4 mM, 0.5 mM, 0.6 mM, 0.667 mM, 0.8 mM, and 1.0 mM of standard VFA mixture. The amount of VFAs in each sample was calculated by comparing the peak area to the standard curve.

Each tested VFA compound was utilized by at least 3 Bacillus strains while heptanoic acid was utilized by all the strains. PTA-3142 and NRRL B-50015 were able to degrade all the tested VFA compounds. Results are recorded in Table 9.

TABLE 9
Average percent reduction of VFAs after 72 h biodegradation

| Accession | | % Reduction of Odor Compounds* | | | | | | |
|---|---|---|---|---|---|---|---|---|
| no. | Identification | IBA | BA | IVA | VA | ICA | CA | HA |
| NRRL B-50014 | Bacillus licheniformis | —** | — | — | 21 | 22 | 26 | 38 |
| NRRL B-59636 | Bacillus mojavensis | — | 31 | — | — | — | 19 | 89 |
| NRRL B-50018 | Bacillus amyloliquifaciens | — | 13 | — | — | 27 | 20 | 95 |
| NRRL B-1001 | Bacillus licheniformis | 97 | 30 | 16 | — | — | 18 | 84 |
| PTA-7790 | Bacillus amyloliquifaciens | — | — | — | — | 34 | 43 | 99 |
| NRRL B-14308 | Bacillus megaterium | 66 | 29 | — | — | — | 76 | 100 |
| PTA-3142 | Bacillus megaterium | 100 | 100 | 100 | 100 | 100 | 100 | 100 |
| PTA-7549 | Bacillus amyloliquifaciens | — | — | — | — | 39 | 46 | 99 |
| NRRL B-50255 | Bacillus pumilus | — | — | — | — | — | 23 | 83 |
| NRRL B-50136 | Bacillus subtilis subsp. subtilis | — | — | — | — | — | 17 | 70 |
| NRRL B-59658 | Bacillus pumilus | — | — | — | — | 21 | 15 | 85 |
| NRRL B-59642 | Bacillus subtilis | — | — | — | — | — | — | 77 |
| PTA-7543 | Bacillus atrophaeus | — | — | — | — | 43 | 20 | 94 |
| NRRL B-50015 | Bacillus licheniformis | 25 | 32 | 17 | 20 | 25 | 27 | 46 |

*All the testes were performed in duplicates and the data indicate averages of two independent measurements. IBA = isobutyric acid; BA = butyric acid; IVA = isovaleric acid; VA -= valeric acid; ICA = isocaproic acid; CA = caproic acid; HA = heptanoic acid
**—No significant reduction.

### Example 5

### Deodorant Spore Stability Study

### Obtaining the Deodorants

A total of six deodorants (no antiperspirants) are used in the stability study. Six of the deodorants were purchased from a retail store and include: Tom's Natural Care of Maine, Old Spice Pure Sport, Axe, Speed Stick, and Right Guard Total Defense Power Deo. The sixth is formulated in-house as shown below:

Deodorant Formulation A

| | Percent Weight | grams |
|---|---|---|
| DiWater | 42.089 | 210.44 |
| Propylene Glycol | 52.611 | 263.06 |
| Sodium Stearate | 5 | 25 |
| Triclosan | 0.2 | 1 |
| Silicone Antifoam | 0.1 | 0.5 |
| | 100 | 500 g |

### Preparation of Spores

NRRL B-50016 and NRRL B-50304 are cultured in 1 liter flasks of Schaeffer's media for one week at 30° C. on a rotary shaker (for Schaeffer's Sporulation Medium, see Schaeffer, P. et al., “Catabolic Repression of Bacterial Sporulation,” Microbiology, 54:704-711 (1965)). The culture is transferred to 1 liter centrifuge bottles and the sample is centrifuged for 20 minutes at 10,000 xg. Being careful not to disturb the pellet, the supernatant is discarded and the pellet is resuspended in 100 mL of deionized water.

### Mixing Spores and Deodorant

Each of the commercial deodorants is removed from its packaging and 75 g of the commercial deodorant is placed in a beaker. Next all of the deodorants, including Deodorant Formulation A, are placed on a hot plate and heated until they reached 80° C. consequently melting the deodorants. Once melted, the deodorants are allowed to cool to approximately 60° C.; the spores are slowly added to the solution and mixed until homogenous.

While the deodorant/spore mixture is still in liquid, it is aliquoted into 1 mL fractions in sealed sterile tubes. One aliquot from each deodorant is retained for immediate testing and to establish counts at time zero while the remaining samples are placed at room temperature (RT), and 35° C. for the stability study.

### Plate Counts

The samples are removed from their respective conditions and 1.0 g of sample is transferred to a 15 mL conical tube containing 9 mL osmolar neutral phosphate buffer. The tubes are then placed in an 80° C. water bath for 10 minutes and plated onto Standard Methods Agar plates (Smith River Biologicals, Ferrum, Va.) using serial dilutions. The plates are then incubated at 35° C. overnight and counts are performed the following morning. Plate counts are conducted once a week for 12 weeks.

The results of this study were summarized in table 10. These data indicated good stability of NRRL B-50016 and NRRL B-50304 over 4 to 12 weeks when stored at RT and 35° C. The spore counts remained the same or dropped less than half of a log for most of the conditions tested. The samples incubated at RT generally have better stability than the samples incubated at 35° C. This study suggest that Bacillus spores survive well in deodorant formulations and thus they may achieve very good shelf-stability with current or modified deodorant formulations.

TABLE 10
Results of Spore Stability in Deodorants

| | Deodorant | | Spore counts after time of incubation | | | | | |
|---|---|---|---|---|---|---|---|---|
| Strain Tested | Formulation | Temperature | 0 | 1 week | 2 weeks | 3 weeks | 4 weeks | 5 weeks |
| NRRL B-50304 | Tom's Natural of | RT | 3.13 × 10⁹ | 1.93 × 10⁹ | 4.27 × 10⁹ | 7.13 × 10⁸ | ND | 9.33 × 10⁹ |
| | Maine | | | | | | | |
| | Speed Stick | RT | 3.93 × 10⁹ | 5.97 × 10⁸ | 2.40 × 10⁸ | 1.24 × 10⁸ | ND | 1.04 × 10⁹ |
| | Right Guard | RT | 1.93 × 10⁹ | 1.63 × 10⁹ | 2.23 × 10⁸ | 5.97 × 10⁸ | ND | 7.40 × 10⁸ |
| | Old Spice Pure | RT | 2.83 × 10⁹ | 2.90 × 10⁹ | 2.37 × 10⁹ | 1.54 × 10⁹ | ND | 2.17 × 10⁹ |
| | Sport | 35° C. | 2.83 × 10⁹ | 3.53 × 10⁹ | 3.83 × 10⁹ | 1.27 × 10¹⁰ | ND | 1.43 × 10⁹ |
| | NZB Formulation | RT | 1.10 × 10⁸ | 1.60 × 10⁷ | 3.37 × 10⁷ | ND | 1.03 × 10⁸ | ND |
| | | 35° C. | 1.10 × 10⁸ | 1.40 × 10⁷ | 8.73 × 10⁷ | ND | 1.20 × 10⁸ | ND |
| NRRL B-50016 | NZB Formulation | RT | 2.07 × 10⁸ | 1.15 × 10⁸ | 2.77 × 10⁸ | 2.87 × 10⁸ | 7.33 × 10⁷ | 6.30 × 10⁷ |
| | | 35° C. | 2.07 × 10⁸ | 1.09 × 10⁸ | 1.90 × 10⁸ | 2.13 × 10⁸ | 9.23 × 10⁷ | 6.57 × 10⁷ |
| | Axe | RT | 2.60 × 10⁸ | 2.88 × 10⁸ | 3.03 × 10⁸ | 4.17 × 10⁸ | 1.77 × 10⁸ | 2.60 × 10⁸ |
| | Old Spice Pure | RT | 2.13 × 10⁸ | 2.09 × 10⁸ | 2.20 × 10⁸ | 2.97 × 10⁸ | 1.83 × 10⁸ | 1.70 × 10⁸ |
| | Sport | 35° C. | 2.13 × 10⁸ | 2.06 × 10⁸ | 1.88 × 10⁸ | 1.40 × 10⁸ | 1.14 × 10⁸ | 9.50 × 10⁷ |

| | Deodorant | | Spore counts after time of incubation | | | |
|---|---|---|---|---|---|---|
| Strain Tested | Formulation | Temperature | 7 weeks | 8 weeks | 9 weeks | 12 weeks |
| NRRL B-50304 | Tom's Natural of | RT | ND | ND | ND | ND |
| | Maine | | | | | |
| | Speed Stick | RT | ND | ND | ND | ND |
| | Right Guard | RT | ND | ND | ND | ND |
| | Old Spice Pure | RT | ND | ND | ND | ND |
| | Sport | 35° C. | ND | ND | ND | ND |
| | NZB Formulation | RT | ND | ND | ND | ND |
| | | 35° C. | ND | ND | ND | ND |
| NRRL B-50016 | NZB Formulation | RT | 3.03 × 10⁷ | ND | 7.93 × 10⁷ | ND |
| | | 35° C. | 2.30 × 10⁷ | ND | 7.97 × 10⁷ | ND |
| | Axe | RT | 1.87 × 10⁸ | ND | 3.33 × 10⁸ | ND |
| | Old Spice Pure | RT | 1.40 × 10⁸ | ND | 1.83 × 10⁸ | ND |
| | Sport | 35° C. | 5.20 × 10⁷ | ND | 6.50 × 10⁷ | ND |

RT: room temperature; ND: not determined

The present invention is described by the following numbered paragraphs:

1. A method of inhibiting production of body malodor caused by bacteria capable of causing body malodor by contacting the bacteria with at least one species of Bacillus or a substance derived from therefrom, wherein the at least one species of Bacillus is selected from the group consisting of Bacillus subtilis, Bacillus amyloliquefaciens, Bacillus pumilus; Bacillus licheniformis, Bacillus megaterium, Bacillus atrophaeus, and Bacillus mojavensis.
2. The method of paragraph 1, wherein the species of Bacillus is Bacillus pumilus.
3. The method of paragraph 1, wherein the bacteria causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.
4. The method of paragraph 1 wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain -NRRL B-59656; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
5. The method of paragraph 4, wherein the at least one strain of Bacillus is selected from the group consisting of Bacillus pumilus strain NRRL B-50016; Bacillus amyloliquefaciens strain NRRL B-50018; Bacillus amyloliquefaciens strain PTA-7543; Bacillus amyloliquefaciens strain PTA-7544; Bacillus amyloliquefaciens strain PTA-7549; Bacillus subtilis strain NRRL B-50136; Bacillus amyloliquefaciens strain NRRL B-50304; Bacillus amyloliquefaciens strain PTA-7790; Bacillus pumilus strain NRRL B-50398; Bacillus pumilus strain NRRL B-50396; and Bacillus pumilus strain NRRL B-50397.
6. The method of paragraph 4, wherein the at least one strain of Bacillus is selected from the group consisting of Bacillus pumilus strain NRRL B-50016; Bacillus pumilus strain NRRL B-50396; and Bacillus pumilus strain NRRL B-50397.
7. The method of paragraph 4, wherein the bacteria causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.
8. A method of inhibiting production of body malodor caused by Corynebacterium mucifaciens by contacting the Corynebacterium mucifaciens with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
9. The method of paragraph 8, wherein the at least one strain of Bacillus is selected from the group consisting of Bacillus pumilus strain NRRL B-50016; Bacillus amyloliquefaciens strain NRRL B-50018; Bacillus amyloliquefaciens strain PTA-7549; and Bacillus amyloliquefaciens strain PTA-7790.
10. The method of paragraph 8, where the at least one strain of Bacillus is Bacillus pumilus strain NRRL B-50016.
11. A method of inhibiting production of body malodor caused by Corynebacterium diphtheriae by contacting the Corynebacterium diphtheriae with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
12. The method of paragraph 11, wherein the at least one strain of Bacillus is selected from the group consisting of Bacillus pumilus strain NRRL B-50016; Bacillus amyloliquefaciens strain PTA-7544; Bacillus amyloliquefaciens strain PTA-7549; and Bacillus subtilis strain NRRL B-50136.
13. The method of paragraph 11, where the at least one strain of Bacillus is Bacillus pumilus strain NRRL B-50016 or Bacillus amyloliquefaciens strain PTA-7549.
14. A method of inhibiting production of body malodor caused by Corynebacterium xerosis by contacting the Corynebacterium xerosis with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
15. The method of paragraph 14, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7543; Bacillus amyloliquefaciens strain PTA-7545; Bacillus amyloliquefaciens strain PTA-7546; Bacillus amyloliquefaciens strain PTA-7549; Bacillus amyloliquefaciens strain PTA-7790; Bacillus amyloliquefaciens strain PTA-7791; and Bacillus amyloliquefaciens strain NRRL B-50304.
16. The method of paragraph 14, where the at least one strain of Bacillus is selected from the group consisting of Bacillus pumilus strain NRRL B-50016; Bacillus amyloliquefaciens strain NRRL B-50018; and Bacillus amyloliquefaciens strain NRRL B-50304.
17. A method of inhibiting production of body malodor caused by Staphylococcus epidermidis by contacting the Staphylococcus epidermidis with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
18. The method of paragraph 17, wherein the at least one strain of Bacillus is selected from the group consisting of Bacillus pumilus strain NRRL B-50016; Bacillus amyloliquefaciens strain PTA-7541; Bacillus amyloliquefaciens strain PTA-7543; Bacillus amyloliquefaciens strain PTA-7544; Bacillus amyloliquefaciens strain PTA-7793; Bacillus subtilis strain NRRL B-50136; Bacillus amyloliquefaciens strain NRRL B-50304; and Bacillus amyloliquefaciens strain NRRL B-50399.
19. The method of paragraph 17, where the at least one strain of Bacillus is Bacillus pumilus strain NRRL B-50016 or Bacillus amyloliquefaciens strain PTA-7543.
20. A method of inhibiting production of body malodor caused by Brevibacterium epidermidis by contacting the Brevibacterium epidermidis with at least one strain of Bacillus or a substance derived from therefrom, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
21. The method of paragraph 20, wherein the at least one strain of Bacillus is selected from the group consisting of Bacillus pumilus strain NRRL B-50016; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7543; Bacillus amyloliquefaciens strain PTA-7544; Bacillus amyloliquefaciens strain PTA-7545; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus amyloliquefaciens strain NRRL B-50304; and Bacillus amyloliquefaciens strain NRRL B-50399.
22. The method of paragraph 20, where the at least one strain of Bacillus is selected from the group consisting of Bacillus pumilus strain NRRL B-50016; Bacillus amyloliquefaciens strain PTA-7543; Bacillus amyloliquefaciens strain PTA-7790; and Bacillus amyloliquefaciens strain NRRL B-50304.
23. A composition adapted for application to the skin of a human comprising at least one species of Bacillus or a substance derived from therefrom, wherein the at least one species of Bacillus is selected from the group consisting of Bacillus subtilis, Bacillus amyloliquefaciens, Bacillus pumilus; Bacillus licheniformis, Bacillus megaterium, Bacillus atrophaeus, and Bacillus mojavensis.
24. The composition of paragraph 23, wherein the species of Bacillus is Bacillus pumilus.
25. The composition of paragraph 23, wherein the composition is a spray or powder.
26. The composition of paragraph 23, wherein the composition is solid or gel composition adapted for application as a deodorant.
27. The composition of paragraph 23, wherein the at least one species of Bacillus is a strain of Bacillus selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
28. The composition of paragraph 27, wherein the at least one strain of Bacillus is selected from the group consisting of Bacillus pumilus strain NRRL B-50016; Bacillus amyloliquefaciens strain NRRL B-50018; Bacillus amyloliquefaciens strain PTA-7543; Bacillus amyloliquefaciens strain PTA-7544; Bacillus amyloliquefaciens strain PTA-7549; Bacillus subtilis strain NRRL B-50136; Bacillus amyloliquefaciens strain NRRL B-50304; Bacillus amyloliquefaciens strain PTA-7790; Bacillus pumilus strain NRRL B-50398; Bacillus pumilus strain NRRL B-50396; and Bacillus pumilus strain NRRL B-50397.
29. The composition of paragraph 27, wherein the at least one strain of Bacillus is selected from the group consisting of Bacillus pumilus strain NRRL B-50016; Bacillus pumilus strain NRRL B-50396; and Bacillus pumilus strain NRRL B-50397.
30. A composition, adapted for application to the feet of a human, comprising at least one species of Bacillus or a substance derived from therefrom, wherein the at least one species of Bacillus is selected from the group consisting of Bacillus subtilis, Bacillus amyloliquefaciens, Bacillus pumilus; Bacillus licheniformis, Bacillus megaterium, Bacillus atrophaeus, and Bacillus mojavensis.
31. The composition of paragraph 30, wherein the at least one species of Bacillus is a Bacillus strain selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
32. The composition of paragraph 30, wherein the at least one strain of Bacillus is selected from the group consisting of Bacillus pumilus strain NRRL B-50016; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7543; Bacillus amyloliquefaciens strain PTA-7544; Bacillus amyloliquefaciens strain PTA-7545; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus amyloliquefaciens strain NRRL B-50304; and Bacillus amyloliquefaciens strain NRRL B-50399.
33. The composition of paragraph 30, wherein the at least one strain of Bacillus is selected from the group consisting of Bacillus pumilus strain NRRL B-50016; Bacillus amyloliquefaciens strain PTA-7543; Bacillus amyloliquefaciens strain PTA-7790; and Bacillus amyloliquefaciens strain NRRL B-50304.
34. A method of inhibiting production of body malodor caused by Brevibacterium epidermidis by contacting the Brevibacterium epidermidis with Bacillus pumilus.
35. The method of paragraph 34, wherein the Bacillus pumilus is at least one strain selected from the group consisting of Bacillus pumilus strain NRRL B-50398; Bacillus pumilus strain NRRL B-59643; Bacillus pumilus strain NRRL B-59644; Bacillus pumilus strain NRRL B-50396; and Bacillus pumilus strain NRRL B-50397.
36. The method of paragraph 34, wherein the Bacillus pumilus is Bacillus pumilus strain NRRL B-50397.
37. A method of inhibiting or preventing the production of body malodor caused by microorganisms capable of producing an odiferous compound(s) comprising subjecting the odiferous compound(s) to at least one bacteria capable of using the odiferous compound as a food source.
38. The method of paragraph 37, wherein the at least one species of bacteria is a species of Bacillus.
39. The method of paragraph 38, wherein the species of Bacillus is selected from the group consisting of Bacillus subtilis, Bacillus amyloliquefaciens, Bacillus pumilus; Bacillus licheniformis, Bacillus megaterium, Bacillus atrophaeus, and Bacillus mojavensis.
40. The method of paragraph 38, wherein the species of Bacillus is Bacillus pumilus.
41. The method of paragraph 37, wherein the microorganism causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.
42. The method of paragraph 38, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain -NRRL B-59656; Bacillus pumilus strain NRRL B-50514; Bacillus pumilus strain NRRL B-50515; Bacillus pumilus strain NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
43. The method of paragraph 37, wherein the odiferous compound is at least one compound selected from the group consisting of short chain fatty acids; e.g. C₂-C₆, and medium chain fatty acids; e.g, C₇-C₁₁.
44. The method of paragraph 43, wherein the short chain fatty acids and medium chain fatty acids are normal, branched, saturated, unsaturated, or any combination thereof.
45. The method of paragraph 37, wherein the odiferous compound is at least one compound selected from the group consisting of acetic acid, isobutyric acid, butyric acid, isovaleric acid, valeric acid, isocaproic acid, caproic acid, heptanoic acid, propionic acid, and octanoic acid.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

We claim:

1. A method of inhibiting production of body maldour caused by microorganisms capable of causing body malodor by contacting the microorganisms with at least one species of bacteria or a substance derived from therefrom.

2. The method of claim 1, wherein the at least one species of bacteria is a species of Bacillus.

3. The method of claim 2, wherein the species of Bacillus is selected from the group consisting of Bacillus subtilis, Bacillus amyloliquefaciens, Bacillus pumilus, Bacillus licheniformis, Bacillus megaterium, Bacillus atrophaeus, and Bacillus mojavensis.

4. The method of claim 2, wherein the species of Bacillus is Bacillus pumilus.

5. The method of claim 1, wherein the microorganism causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.

6. The method of claim 2, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain -NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.

7. A method of inhibiting or preventing the production of body malodor caused by microorganisms capable of producing an odiferous compound(s) comprising subjecting the odiferous compound(s) to at least one bacteria capable of using the odiferous compound as a food source.

8. The method of claim 7, wherein the at least one species of bacteria is a species of Bacillus.

9. The method of claim 8, wherein the species of Bacillus is selected from the group consisting of Bacillus subtilis, Bacillus amyloliquefaciens, Bacillus pumilus; Bacillus licheniformis, Bacillus megaterium, Bacillus atrophaeus, and Bacillus mojavensis.

10. The method of claim 8, wherein the species of Bacillus is Bacillus pumilus.

11. The method of claim 7, wherein the microorganism causing the malodor is at least one bacterium species selected from the group consisting of Corynebacterium mucifaciens; Corynebacterium diphtheriae; Corynebacterium xerosis; Staphylococcus epidermidis; and Brevibacterium epidermidis.

12. The method of claim 8, wherein the at least one strain of Bacillus is selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain -NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.

13. The method of claim 7, wherein the odiferous compound is at least one componud selected from the group consisting of short chain fatty acids; e.g. C₂-C₆, and medium chain fatty acids; e.g, C₇-C₁₁.

14. A composition adapted for application to the skin of a human comprising at least one species of bacteria or a substance derived from therefrom.

15. The composition of claim 14, wherein the at least one species of bacteria is a species of Bacillus.

16. The composition of claim 15, wherein the at least one species of Bacillus is selected from the group consisting of Bacillus Bacillus subtilis, Bacillus amyloliquefaciens, Bacillus pumilus; Bacillus licheniformis, Bacillus megaterium, Bacillus atrophaeus, and Bacillus mojavensis.

17. The composition of claim 15, wherein the species of Bacillus is Bacillus pumilus.

18. The composition of claim 14, wherein the composition is a spray or powder.

19. The composition of claim 14, wherein the composition is solid or gel composition adapted for application as a deodorant.

20. The composition of claim 15, wherein the at least one species of Bacillus is a strain of Bacillus selected from the group consisting of Bacillus pumilus strain NRRL B-50016; Bacillus amyloliquefaciens strain NRRL B-50017; Bacillus amyloliquefaciens strain NRRL B-50018; Bacillus amyloliquefaciens strain PTA-7541; Bacillus amyloliquefaciens strain PTA-7792; Bacillus amyloliquefaciens strain PTA-7542; Bacillus amyloliquefaciens strain PTA-7543; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136; Bacillus amyloliquefaciens strain NRRL B-50304; Bacillus amyloliquefaciens strain NRRL B-50399; Bacillus pumilus strain NRRL B-50398; Bacillus pumilus strain NRRL B-59643; Bacillus pumilus strain NRRL B-59644; Bacillus pumilus strain NRRL B-50396; Bacillus pumilus strain NRRL B-50397; Bacillus pumilus strain NRRL B-50014; Bacillus pumilus strain NRRL B-50255; Bacillus licheniformis strain NRRL B-1001; Bacillus megaterium strain NRRL B-14308; Bacillus megaterium strain PTA-3142; Bacillus amyloliquifaciens strain NRRL B-59658; Bacillus mojavensis strain NRRL B-59636; Bacillus mojavensis strain -NRRL B-59656; Bacillus pumilus strain NRRL B-50514; Bacillus pumilus strain NRRL B-50515; Bacillus pumilus strain -NRRL B-59651; Bacillus pumilus strain NRRL B-59652; Bacillus pumilus strain NRRL B-59655; Bacillus amyloliquifaciens strain NRRL B-59657; Bacillus amyloliquifaciens strain NRRL B-59647; Bacillus amyloliquifaciens strain NRRL B-59649; Bacillus amyloliquifaciens strain NRRL B-59650; Bacillus amyloliquifaciens strain NRRL B-59653; Bacillus subtilis strain NRRL B-59651; Bacillus subtilis strain NRRL B-59648; Bacillus subtilis strain -NRRL B-59654; and Bacillus subtilis strain NRRL B-59642.
